(19) United States
(12) Patent Application Publication
(10) Pub. No.: US 2009/0043300 A1
(43) Pub. Date: Feb. 12, 2009

(54) APPARATUS AND METHOD FOR CLEARANCE CALIBRATION OF SHOCK WAVE ELECTRODES

(75) Inventors: Ralph Reitmajer, Konstanz (DE); Reiner Schultheiss, Illighausen (CH); Rudiger Bolze, Konstanz (DE)

Correspondence Address: DAVID L. KING, SR., 5131 N.E. COUNTY ROAD 340, HIGH SPRINGS, FL, 32643, US

(73) Assignee: MTS Europe GmbH, Konstanz (DE)

(21) Appl. No.: 12/104,512

(22) Filed: Apr. 17, 2008

(30) Foreign Application Priority Data
Apr. 17, 2007 (DE) 10 2007 018 841.4

(51) Int. Cl. A61B 18/14

(52) U.S. Cl. 606/41

(57) ABSTRACT

The invention is for an apparatus for the generation of shock waves, especially for medical application, by means of a spark discharge between two electrodes (6, 7) within a liquid medium with appliances for the movement (216) of at least one of the electrodes (6, 7) along a specified direction of movement, for an electrode unit for the generation of shock waves with two electrodes (6, 7) positioned within a liquid medium and to which a high voltage can be applied for the generation of an electrical disruptive discharge, for a procedure for the calibration of distances for a pair of electrodes (1; 201) for the generation of shock waves by means of an under water spark discharge and for a procedure for the generation of shock waves by means of an under water spark discharge between two electrodes (6, 7).

Reasonable distances of the electrodes (103) may be determined within a pre-defined scope, if appliances are available for the limitation of the travel (210) of et least one of the electrodes between a first limit position that corresponds to the smallest possible distance (3) between the two electrodes, and a second limit position that corresponds to the largest possible distance (103) between the electrodes.

The invention is for an apparatus for the generation of shock waves, especially for medical application, by means of a spark discharge between two electrodes within a liquid medium with appliances for the movement of at least one of the electrodes along a specified direction of movement, for an electrode unit for the generation of shock waves with two electrodes positioned within a liquid medium and to which a high voltage can be applied for the generation of an electrical disruptive discharge, i.e. an electrical disruptive discharge through the tips of electrodes, for a procedure for the calibration of distances for a pair of electrodes for the generation of shock waves by means of an under water spark discharge and for a procedure for the generation of shock waves by means of an under water spark discharge between two electrodes.

Prior solutions include appliances where under water spark discharge is used for generation of shock waves, i.e., for non-contact destruction of concrements in bodies of living beings. The discharge is made through a spark gap between two electrodes that are located in the focus of a reflector. Inventions to resolve the electrode burn-off at each discharge include appliances for generation of shock waves by means of a spark gap where the electrodes can be replaced. DE 33 16 837 C2, for example, presents an appliance in which the electrodes can be shifted against each other by rotating a sleeve. U.S. Pat. No. 4,868,791 and U.S. Pat. No. 5,208,788 disclose procedures which can be used to bring tips of electrodes in a pre-selected distance. To this end, each tip of the electrodes is first lead to a contact sensor which is then approached to the focal point of the reflector for alignment and finally removed from it by a particular distance.

EP 0 911 804 further discloses a procedure in which the electrode distance can be adjusted during operation according to the discharge characteristics.

The operating life for apparatuses with electrodes without distance replacement ends when the electrode distance becomes too large and an applied voltage does not cause a discharge anymore. However, the operating life for apparatuses providing a replacement of the electrodes is subject to the mechanically possible advance distance and/or the amount of the electrode material which can be replaced.

In either case, the end of the operating life is signified to the user in that the intended use is not possible anymore if, for example, occurrence of an increased number misfirings.

As it is not desirable to reach the range with reduced shock wave quality or constancy, especially in medical applications, the operation of the electrodes must be constantly monitored. The shock wave quality can be characterized, for example, by the pressure profile, especially the steepness of the pressure or the proportion of pressure and tension. The constancy of the shock waves refers to a repeatability of the pressure profile from shot to shot and the diversification of the pressure profile parameters.

The quality and the constancy can be ensured by using pairs of electrodes for a certain number of discharges only, also referred to as number of shots. In such case the pairs of electrodes are generally not used fully.

A problem can also occur if the operation is interrupted, for example, by the end of a medical treatment of a patient and restarted at a later point with the pair of electrodes that have already been used. The number of shots per pair of electrodes can indeed be stored. However, apparatuses known from prior solutions do not allow an objective determination of the condition of wear in an installed condition.

Storing the number of shots does not provide a possibility to determine during the shock wave operation and while the pair of electrodes is actually firing if the tip of electrodes is damaged, e.g., broken.

The aim of the invention is to overcome disadvantage of the prior art and to introduce apparatuses and procedures allowing definition of reasonable distances of the electrodes within a pre-defined scope.

The aim is attained by an apparatus and a procedure with the features of the independent claims.

First, the aim is attained by an apparatus with the features of Claim No. 1. The apparatus features appliances for limiting the travel of at least one of the electrodes between a first limit position that corresponds to the smallest possible distance between the two electrodes, and a second limit position that corresponds to the largest possible distance between the electrodes. Therefore, at least one of the electrodes can be moved on a line between the limit positions only and thus the limit positions define the scope for the travel and the possible distance between the electrodes. The distance for the movement of at least one of the electrodes preferably has a monotonous path.

By limiting the travel it can be avoided that one of the electrodes is removed from the reflector, that the electrodes are separated completely, that the electrodes collide or are pushed past each other, during operation.

In general, the electrodes have a pin or wire type shape and are arranged axially with two tips of electrodes facing each other. In such a case, the distance between the electrodes refers to the distance of the tips of the electrodes.

If an unlimited amount of electrode material can be approached the initially possible distances of electrodes can be generated over a long operation period. In general, however, the length of the electrodes is limited. The burn-off causes a further decrease of the length which results in a change of the possible distances and thus the possible limit positions, during the operation period.

Subject to the arrangement and the travel mechanics of the electrodes in the reflector it may happen that as a result of the burn-off the smallest possible distances or the largest possible distances constantly increase. In both cases the possible travel distance is an indicator for the wear or the “age” of the electrode.

A limit position can be defined in that a reference point on the electrode or on the electrode support reaches a certain position in respect to the other electrode ox the reflector. A limit position can be defined by a reference position that, for example, passes a light barrier and creates, interrupts or measurably changes an electrical connection.

A reference point is preferably variable and changes its position with decreasing length of the electrode. It can be defined, for example, by the outermost tip of the electrodes.

In an advantageous embodiment of the invention the first limit position and/or the second limit position are determined by a mechanical stop.

There are several ways to detect if the mechanical stop is reached, for example, by an electrical signal.

The stop can also be used to mechanically block further travel. A mechanical stop does not only define the limit position but can also prevent the travel from continuing beyond the limit position.

Particularly advantageous is a procedure arrangement in which the first limit position is given by a contact of the tips of electrodes, especially throughout the entire operating life of the electrode.

The distance reached in the first limit position can then be used as a reference distance and as a zero point for a distance evaluation.

In general, an assembly of electrodes in which the electrodes can be approached without limitations throughout the entire operation period is advantageous since working with small voltages is only possible for small distances while generation of larger discharge voltages is also possible with larger electron distances. Thus, the intensity of the shock waves which is immediately linked to the discharge voltage can be adjusted throughout the entire operation period, if selection of small distances is possible at any time.

In a preferred embodiment, of the invention the apparatus is equipped with appliances for measuring the travel distance. The distance can be determined, for example, based on the time which is needed for the travel between the limit positions at a constant speed, on a number of rotations of an operating mechanism, on the number of pulses of an incremental encoder or by means of a measurement of the distance between the positions at the limit positions of a reference point.

The user can relate the current travel distance to the length of the electrodes and the remaining electrode material. The change of the travel distance throughout the operating time is an indicator for the wear of the electrodes. The travel distance further reveals the possible distances of the electrodes. The user can make conclusions with respect to the voltage which needs to be applied in order to reach a disruptive discharge.

Another advantageous embodiment of the invention is that the apparatus is equipped with appliances for determination of the electrode distance. This is especially advantageous if a pair of electrodes is recommissioned after already being used since the distance of the electrodes changes due to the use. For example, it can be determined if the burn-off occurred to the expected extent.

The cause for deviations from the expected behaviour can be, for example, the damage of an electrode which can be determined by means of a distance measurement.

If the first limit position is defined by the contact of the tips of electrodes, the distance of the electrodes in an actual position can be determined by identifying a part of the entire possible travel, i.e. the distance of travel from the actual position to the first limit position.

In an advantageous embodiment the apparatus is equipped with appliances for determination of the remaining length of the electrode.

The assembly of the electrodes reveals the possible maximum travels for unused electrodes. The difference between a maximum distance of travel determined at a certain time and the maximum distance of travel for unused electrodes can be used for determination of the burn-off or the remaining length of electrodes. Theses values provide information on the number of discharges which can be generated with the pair of electrodes. This value is referred to as remaining number of shots.

The burn-off of the electrodes and thus the possible number of shots is subject to the electrode material, especially the composition of the alloy, the hardness, the conductivity, the elasticity, etc. The burn-off is also subject to the liquid in which the electrodes are positioned.

Preferably, the length of electrodes or the remaining number of shots is determined automatically by means of an evaluation unit.

The remaining length of electrodes according to the number of shots released can be used to determine if the burn-off corresponds to the expectations or if a damage of the electrodes occurred.

The invention is developed in an advantageous manner if the apparatus is equipped with appliances for setting a specific electrode distance.

The distance of the electrodes is correlated with the distance of a reference point of one of the limit positions. The setting of the distance is particularly easy if the first limit position is defined by the contact of the electrodes. At least one electrode that can be travelled is simply to be removed, from the first limit position by the required length.

At first determination of the entire possible travel can reveal if the desired, distance of electrodes can be realized at all for the existing burn-off. If this is not the case a corresponding signal output can be made.

The user can use the selection of the distance for pre-selection of an optimal distance value for the generation of a shock wave with a defined energy or for a discharge at a defined discharge voltage. The setting of the distance can also be used in connection with an automated readjustment of the electrodes. The setting of the distance can be used for performance of a coarse adjustment and/or can be part of the adjustment mechanism.

The selection of optimized distances of electrodes is made, for example, by means of an evaluation of the discharge curve or the pressure profile. A table can be prepared showing the optimum distances for each voltage. At the beginning of a discharge series a relevant value can be extracted from the table. The distance can be readjusted during operation based on a control of the discharge curve. The appliances for measuring the travel distance can be used for continuous control also during readjustment if due to the readjustment one of the limit positions is reached and if further discharges are possible.

A preferred embodiment of the invention is obtained if an operating mechanism, especially a motor or a stepper motor is provided for the travel of at least one of the electrodes. An operating mechanism with a measurable movement allows a repeatable and precise setting of the position of the electrodes.

Possible operating mechanisms are, for example, a stepper motor, a linear motor, a servomotor, a piezo motor, a pneumatic, a hydraulic or a different operating mechanism or adjusting mechanism.

Advantageously, an incremental encoder, a resolver or an absolute-value encoder is provided for the activation and control of the operating mechanism.

The control signals, for example, the number of the increments until a mechanical stop is reached can be used as indicator for the travelled length.

The operating mechanism can preferably be connected to at least one of the electrodes through a gear drive. If, for example, the displacement of the electrodes is realized by means of a rotational movement of a thread in respect to a mating thread the gear drive can adjust the rotation of the motor to the rotation of the electrodes.

The inner conductor electrode that is driven by the rotation of the motor rotates in accordance with the thread pitch, the motor speed, and the gear drive.

Alternatively, the inner conductor and the outer conduct or may be coupled through a thread and simultaneously move against each other. Varying burn-off characteristics of the two electrode tops cars be compensated by varying thread transmissions.

In an advantageous embodiment of the apparatus appliances for generating and reading out messages with respect to travel, possible distance values between the electrodes, and/or the functionality of the pair of electrodes is provided.

This may either be an optical or acoustic alarm signal which is read out if s selected limit value is reached or exceeded. For example, a selected distance of electrodes can not be set if the possible travel is below or above a defined value or the determined length of electrodes is below a limit value. Also possible is the indication of one or several measured or determined values on a display, such as, for example, the remaining length of electrodes, the remaining operating life of the electrode, measured in an expected number of shots. Alternatively or additionally, a recommendation for a defined voltage range of the discharge voltage between the electrodes can be displayed.

The aim is further attained by an electrode unit, especially for medical application, comprising two electrodes, particularly positioned within a liquid medium, to which high voltage can be applied in order to generate an electrical disruptive discharge. At least one of the electrodes can travel between a first limit position that corresponds to the smallest possible distance between the two electrodes and a second limit position that corresponds to the largest possible distance between the electrodes, and the apparatus is equipped with appliances for determining the first and the second limit position.

In an apparatus as described above a pair of electrodes can especially be used for generation of shock waves.

The ability to travel between the two limit positions ensures that the electrodes cannot drift apart from each other, i.e. the pair of electrodes cannot be separated.

Apart from the two electrodes, the apparatus features appliances for determination of the limit position. The electrodes can be arranged such that the limit positions between which an electrode can be travelled are defined independently from the reflector to which the pair of electrodes is mounted. The pair of electrodes which can be replaced can also be mounted in various devices and/or various reflectors in which case the possible distance of travel is a feature of the pair of electrodes and its wear and does not depend on the device or the reflector.

If required, the limit positions can be determined such that they are adapted to a cert a in device or reflector geometry.

As already described, above, the determination of a limit position can be made by means of detection of a first reference point in respect to a second reference point.

Advantageously, the first limit position and/or the second limit position are determined by a mechanical stop. A mechanical stop serves as a signal for the limit position and can simultaneously block the travel from being continued.

Alternatively, the limit position can be defined by releasing an electrical or optical switch or by the signal of a light barrier, a pneumatic cylinder, a pressure sensor or an induction switch.

The limit position that corresponds to the smallest possible electrode distance is preferably defined by a contact of the tips of the electrodes. The limitation of the travel distance at this limit position prevents the electrodes from being damaged or that the electrodes are pushed past each other. At the same time the contact of the tips of the electrodes corresponds to the smallest possible distance between the electrodes and can therefore be used as sera point for a distance evaluation.

In general, the voltage supply of the electrodes de by means of a co-axial cable, said electrodes being connected to the outer conductor and one of the electrodes to the inner conductor. In an advantageous embodiment of the invention the electrode that is connected to the inner conductor and arranged in a way that it can be moved and replaced.

The electrode is attached to an inner conductor pin.

The second limit position is preferably determined by a contact of the inner conductor pin with a contact surface. The contact surface has a defined position in respect to a reference point on the other electrode or in respect to the outer conductor connection.

The electrode unit itself can be equipped with operating appliances for travelling of at least one electrode. In an advantageous embodiment the electrode unit is equipped with a coupling mechanism for connecting the apparatus to another apparatus which in turn is equipped with appliances for moving at least on of the electrodes, particularly to an apparatus as described above.

The appliances for determination of the limit positions can act on the operating appliances through the coupling mechanism.

The aim is further attained by a procedure for calibration of distances for a pair of electrodes for the generation of shock waves by means of an under water spark discharge, particularly in an apparatus as described above with the following steps of procedure. At first a travel of at least one electrode is performed to a minimum distance between the electrodes. Then at least one electrode is travelled to a maximum, distance between the electrodes. Simultaneously,, the distance of travel is measured. The steps of procedure can also be performed in reverse order, i.e. first the largest distance is taken and then the smallest distance.

The distance of travel is an indicator for the current condition of the pair of electrodes. The travel from the smallest to the largest possible distance covers all currently possible distances.

In a subsequent step a certain pre-selected distance can be set provided that it is within the interval. It is thus favourable if the minimum possible distance coincides with a contact of the tip of the electrodes. The distance of travel can then correspond to the selected distance. Alternatively, the minimum possible distance between the electrodes must be considered in addition.

Moreover, the calibration of distances can be used to determine the remaining length, of electrodes and especially the remaining number of shots in a subsequent step. To this end, the maximum distance of travel is compared to the corresponding maximum length of the distance of travel of a similar used pair of electrodes or to the theoretically possible maximum distance of travel. As a certain burn-off of the electrodes is to be expected for every discharge the remaining lengths of electrodes are directly related to the remaining number of possible discharges, i.e. the remaining number of shots.

When starting to use a pair of electrodes determination if it is a new pair of electrodes or a pair of electrodes that has already been used is possible by measuring the maximum length of the distance of travel.

As the calibration of distances delivers a result with respect to the condition of the pair of electrodes it is advantageous if in a subsequent step a message with respect to the functionality of the pair of electrodes is read out. In particular, the user can be warned if the operating life of the pair of electrodes approaches its end, the end of the operating life has already been reached, certain distances have not been selected yet or cannot be selected anymore or a damage of the pair of electrodes is suspected. The message can be communicated by means of display of a measurement value or emission of an optical or acoustic signal.

The aim is further attained by a procedure for the generation of shock waves by means of an under water spark discharge between two electrodes, particularly in an apparatus as described above with the following steps of procedure.

At first, a calibration of distances is made and the remaining number of shots is determined, especially according to one of the procedures described above. Subsequently, the required energy level and the required number of shots is selected. In general, the energy level is selected, by definition of a voltage which needs to applied to the electrodes for the discharge.

The corresponding distance of the electrodes is determined prior to verification if the distance of electrodes and the required number of shots is compatible with the remaining number of shots that was determined during the calibration of distances. Eventually, an error message is read out and/or the procedure aborted. In a subsequent step, the distance between the two electrodes is selected. The required number of discharges can now be generated. The steps of procedure are either repeated from the calibration of distances or from the selection of the energy level.

In addition, after every discharge or after a series cf discharges a readjustment of the distance between the two electrodes can be executed. The readjustment process can be influenced by characteristic properties of the discharge curve, e.g., by the ignition delay periods, the disruptive discharge voltage, the amount of the flowed charge, the maxima of the current and/or voltage curves, the zero transitions of the current and/or voltage curves, the pressure profile, and/or the remaining measured values at the electrical disruptive discharge or due to the electrical discharge. (Observation of the discharge)

The readjustment can either be made exclusively based on a table in which the burn-off of the electrodes under consideration of the used energy levels and the quality of the electrodes is included statistically or exclusively based on the observation of the discharge. The two procedures for readjustment can be combined allowing, for example, to control and correct the readjustment by means of statistical determination of the wear through observation of the discharge.

Especially for generation of longer series of discharge, it makes sense to check the functionality of the pair of electrodes not only between the individual discharge series but also during the series. Advantageously, a calibration of distances is executed after a pre-defined number of discharges, for example, on regularly basis after the same number of discharges. The calibration of distances is especially executed according to a procedure as described above.

The invention, its usefulness, and additional benefits are explained based on the drawings described below.

### DESCRIPTION OF THE DRAWINGS

FIG. 1a is a schematic representation of an unused pair of electrodes with maximum possible distance of the tips of electrodes;

FIG. 1b is a schematic representation of the unused pair of electrodes with minimum possible distance of the tips of electrodes;

FIG. 2a is a schematic representation of a used pair of electrodes that already experienced a certain burn-off with maximum possible distance of the tips of electrodes;

FIG. 2b is a schematic representation of the used pair of electrodes that already experienced a certain burn-off with minimum possible distance of the tips of electrodes;

FIG. 3 is a schematic representation of a pair of electrodes with appliance for limiting the travel and operating appliances;

FIG. 4a is a sectional view of an unused pair of electrodes with movable inner conductor electrode;

FIG. 4b is a sectional view of a used pair of electrodes with movable inner conductor electrode;

FIG. 5a is a sectional view of an unused pair of electrodes with movable electrodes;

FIG. 5b is a sectional view of a used pair of electrodes with movable electrodes;

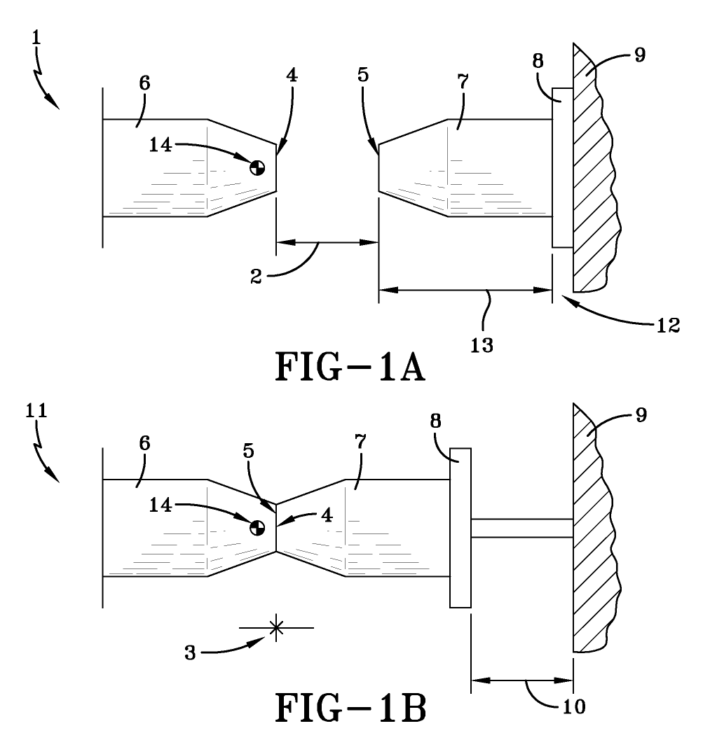

FIGS. 1a and 1b are schematic representations of a new pair of electrodes 1 with maximum possible distance 2 and minimum possible distance 3 of the tips of electrodes 4, 5.

In the embodiment described one of the electrodes 6 is arranged fixedly. This electrode 6 is preferably connected to the outer conductor that is not shown in the figure. The remaining electrode 7 is movably mounted and is connected to the inner conductor that is not explicitly shown in the figure through the inner conductor pin 8. The maximum possible distance 2 between the electrodes 6, 7 is reached when the inner conductor pin 8 contacts a contact surface. The smallest possible distance 3 between the electrodes 6, 7 is defined by the contact of the tips of electrodes 4, 5 in which case the distance is zero. The movable electrode 7 can be moved between two limit positions 11, 12, the said limit position 11 being defined by the contact of the tips of electrodes 4, 5 and the other limit position 12 by the mechanical contact of the inner conductor pin 8 with the contact surface 9.

Prior to their initial use the electrodes 6, 7 have an initial length 13. The electrodes 6, 7 are arranged such that the focal point 14 of the reflector that is not shown in the drawing is located within the electrodes 6, 7 or the distance 2. In FIG. 1 it is exemplarily located within the unmoved electrode 6.

After use the electrodes 6, 7 become smaller due to the burn-off.

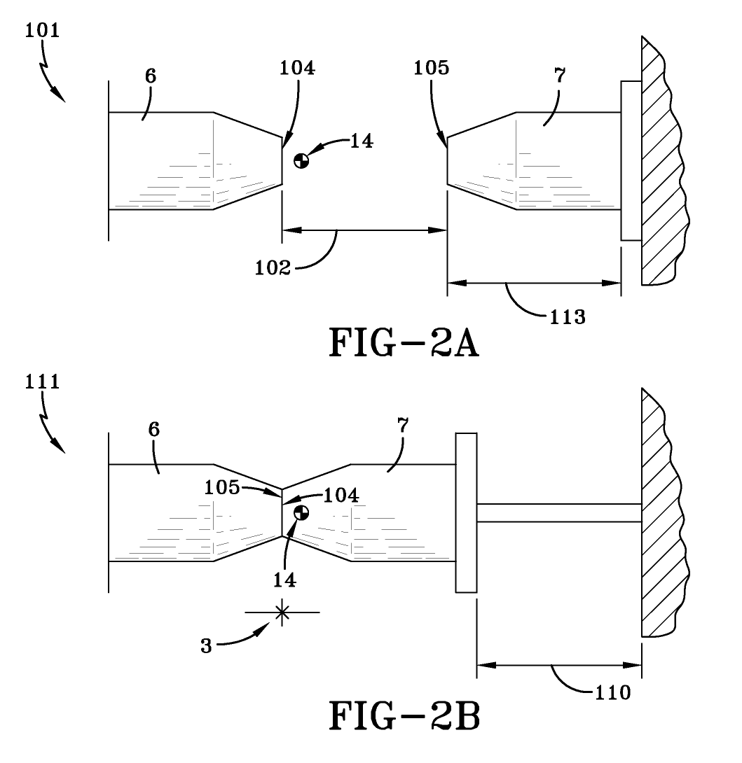

FIGS. 2a and 2b are schematic representations of a used pair of electrodes 101 that already experienced a certain burn-off with maximum possible distance 102 and minimum possible distance 3 of the tips of electrodes 104, 105.

The length of electrodes 113 was reduced by the burn-off such that the focal point 14, for example, may be located outside the electrode 6. During the operating life of the pair of electrodes 1 the discharge is thus always made in immediate vicinity of the focal point 14.

The shortening of the electrodes 6, 7 results in a larger maximum distance 102 of the tips of electrodes 104, 105 and thus in a larger travel 110. The travel 110 is increased, compared to the possible original travel 10 by the length of burn-off of one electrode 6 and the length of burn-off of the other electrode 7. Since it is assumed that the two electrodes 6, 7 burn off proportionally the change of the maximum travel 110 indicates the length 113 of the electrodes 6, 7 and thus the number of the remaining possible discharges.

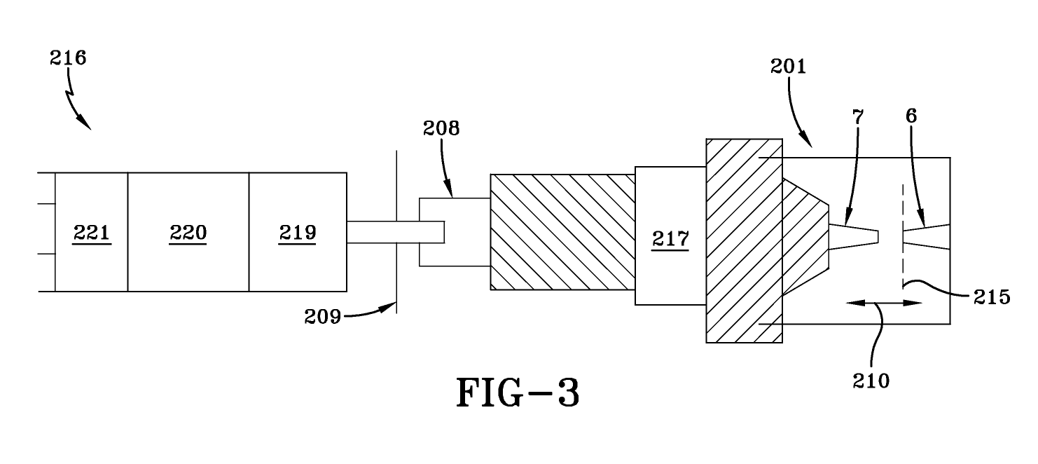

FIG. 3 is a schematic representation of a pair of electrodes 201 with appliances 209, 215 for limitation of the travel 210 and operating appliances 216;

The electrode 7 which is connected to the inner conductor is arranged movably. The travel 210 is limited on one side in such way that the inner conductor pin 208 contacts the rear contact surface 209 end on the other side in such way that the movable electrode 7 is subject to a mechanical stop 215 at the unmovable electrode 6.

The unmovable electrode is connected to the outer conductor 217.

The operating mechanism of the electrode 7 that is mounted on the inner conductor pin 208 is realized by a threaded rod 218 that is connected to a motor 220 through a gear drive 219. The motor, in turn, is monitored by a generator 221 (i.e. pulse generator, resolver, incremental encoder, etc.).

If the electrode 7 contacts the mechanical stop 215 the power consumption of the motor 220 increases. If the power consumption exceeds a previously defined threshold value the operating mechanism is stopped.

In an exemplary assembly the pulse generator 220 emits 2×16 pulses that reveal a phase shift each. The rising and the failing edge are detected in such way that two incremental steps are performed per pulse. Thus (64(=16×2×2) incremental steps occur per rotation of the motor. The used gear drive 219 has a reduction ratio of 152:1.

The inner conductor pin 208 comprises an M5 thread with a pitch of 0.8 mm per rotation.

An advance of 0.8 mm is performed for each rotation of the motor which corresponds to a number of 9728 incremental steps. On the other hand 12169 incremental steps correspond to an advance of 1 mm.

For an initial distance between the tips of electrodes of 1.05 mm and a maximum admissible burn-off of an electrode of 4 mm the inner conductor electrode must have a maximum travel of 9.85 mm which is generated by 123125 incremental steps.

The number of incremental steps for a displacement of the inner conductor electrode from one end point to the opposite stop allows determination of the travel and indicate the remaining length of electrodes.

An alarm signal can be read out if the travel exceeds or equals 9.85 mm, i.e. the maximum admissible distance is reached.

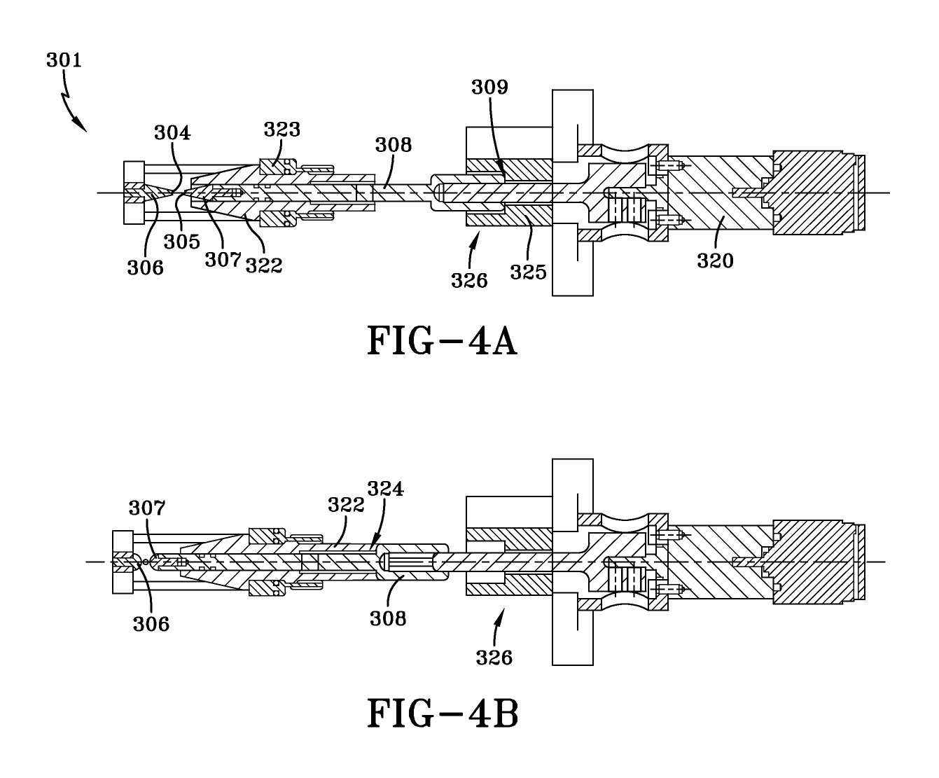

FIG. 4a is a sectional view of an unused pair of electrodes 301 with movable inner conductor electrode 307. The inner conductor electrode 307 is connected to the inner conductor pin in a conductive fashion. The inner conductor is movably mounted in a fixed isolator 322. The isolator 322 and the outer conductor 323 for the outer conductor electrode 306 are located opposite the reflector that is not shown in the drawing in a stationary position. The inner conductor electrode 307 can be moved by means of a movement of the inner conductor pin 308. Until the electrodes 306, 307 are not entirely burned off the first limit position corresponding to the smallest possible distance between the electrodes is reached by means of a stop that is not explicitly shown in the drawing when contacting the tips of electrodes 304, 305. However, if the electrodes 306, 307 as shown in FIG. 4b are entirely burnt off the first limit position is reached by means of a contact of the inner conductor pin 308 with a contact surface 324 of the isolator 322.

The second limit position in which the tips of electrodes have the largest possible distance is defined by a contact of the inner conductor pin 308 with a contact surface 309 in the filling piece 325 of the high voltage connection 326.

The described contacts that correspond to the minimum and maximum possible distances of the tips of electrodes 306, 307 limit the travel of the inner conductor pin 308.

The operating mechanism of the inner conductor pin 308 is realized by means of a geared motor 320.

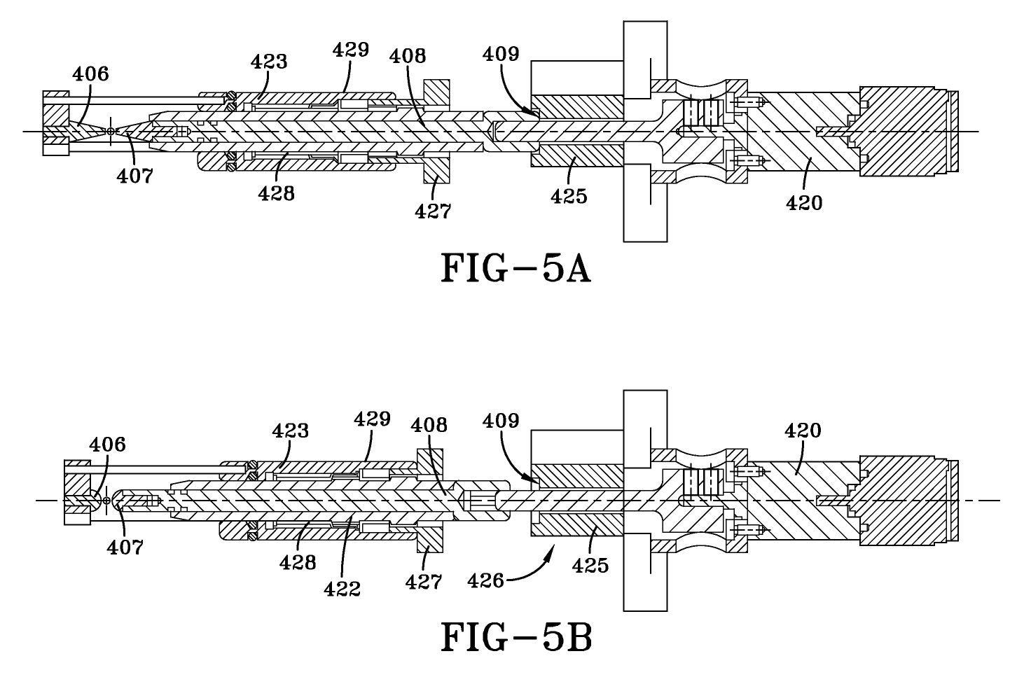

In an alternative embodiment the two electrodes 406, 407 are movable, as shown in the FIGS. 5a and 5b. In this case the outer conductor electrodes 406 and the outer conductor 423 are not stationary in respect to the reflector that is not shown in the figure but can be moved in respect to the electrode base 427.

The inner conductor electrode 407 is attached to the inner conductor pin 408 that can be moved together with the isolator 422 in respect to the outer conductor 403 that is movable. If the inner conductor pin 408 is removed from the operating mechanism 420 a threaded connection 428 and a locking device 429 between outer conductor 423 and electrode base 427 ensures that the outer conductor 423 simultaneously moves towards the electrode base 427. In such way the electrodes 406, 407 approach each other.

The inner conductor pin 408 can be moved towards the geared motor 420 until it contacts a contact surface 409 in the filling piece 425 of the high voltage connection 426. If the inner conductor pin 408 is travelled in the direction of the geared motor 420 the threaded connection 428 causes the outer conductor 423 to be removed from the electrode base 427 which causes the outer conductor electrode 406 to move. If the inner conductor pin 408 contacts the contact surface 409 the electrodes 406, 407 have their maximum possible distance.

If the electrodes 406, 407 are not burnt off yet they can be approached until a stop is caused by a contact of the tips of electrodes. However, if the electrodes 406, 407 are burnt off the mutual travel of inner conductor pin 408 and outer conductor 423 is stopped when the isolator 422 that is moved with the inner conductor pin 408 contacts the electrode base 427. In either case the first limit position that corresponds to the minimum possible distance between the electrodes 406, 407 for a defined length of electrodes 406, 407 is reached.

### REFERENCE SIGNS

Pair of electrodes 1; 101; 201
Minimum possible distance 3; 103
Maximum possible distance 2; 102
Tips of electrodes 4, 5; 104, 105; 304, 305
One of the electrodes 6; 306; 406
Other electrode 7; 307; 407
Inner conductor pin 8; 208; 308, 408
Contact surface 9; 209, 309; 409
Travel 10; 110; 210
Limit positions 11, 12
Initial length 13
Focal point 14
Appliance for limiting the travel 215
Operating appliances 216
Outer conductor 217
Screw driver 218
Gear drive 219
Motor 220
Pulse generator 221
Isolator 322
Outer conductor 323
Contact surface 324
filling piece 325
High voltage connection 326
Electrode base 427
Threaded connection 428

1. Apparatus for the generation of shock waves, especially for medical application, by means of spark discharge between two electrodes within a liquid medium, including appliances for moving at least one of the electrodes along a specified direction of movement, with the apparatus featuring appliances for limiting the travel of the electrode between a first limit position that corresponds to the smallest possible distance between the two electrodes, and a second limit position, that corresponds to the largest possible distance between the electrodes.

2. Apparatus according to claim No. 1, wherein the first limit position and/or the second limit position is/are determined by a mechanical stop, and especially the first limit position is determined by the contact of the electrode tips.

3. Apparatus according to claim No. 1, wherein the first limit position is determined by a reference position that passes a light barrier and creates, interrupts or measurably changes an electrical connection,

4. Apparatus according to claim No. 2, wherein the apparatus is equipped with appliances for measuring the travel distance.

5. Apparatus according to claim 2 wherein the apparatus is equipped with appliances for determining the electrode distance.

6. Apparatus according to claim 2 wherein the apparatus is equipped with appliances for determining the remaining length of the electrodes.

7. Apparatus according to claim 2 wherein the apparatus is equipped with appliances for setting a specific electrode distance.

8. Apparatus according to claim 2 wherein an operating mechanism, especially a motor or a stepper motor is provided for the actuation of at least one of the electrodes.

9. Apparatus according to claim No. 8, wherein an incremental encoder, a resolver or an absolute-value encoder is provided for the activation and control of the operating mechanism.

10. Apparatus according to claim No. 9, wherein the operating mechanism can be connected to at least one of the electrodes via a gear drive.

11. Apparatus according to claim 9 wherein the equipment for generating and reading out messages with respect to travel, possible distance values between the electrodes and/or the functionality of the pair of electrodes is provided.

12. Electrode unit for the generation of shock waves, especially for medical application, comprising of two electrodes—particularly positioned within a liquid medium—to which high voltage can be applied in order to generate an electrical disruptive discharge, distinguished by the characteristic feature that at least one of the electrodes can travel between a first limit position that corresponds to the smallest possible distance between the two electrodes, and a second limit position, that corresponds to the largest possible distance between the electrodes and that the apparatus is equipped with appliances for determining the first and the second limit position.

13. Apparatus according to claim No. 12, wherein distinguished by the characteristic feature that the first limit position and/or the second limit position is/are determined by a mechanical stop, and especially the first limit position is determined by the contact of the electrode tips, and especially the second limit position is determined by the contact of a central conductor pin with a stop face.

14. Apparatus according to claim No. 13, wherein the apparatus is equipped with a coupling mechanism for connecting the apparatus to another apparatus that in turn is equipped with appliances for moving at least one of the electrodes.

15. Electrode unit for the generation of shock waves, especially for medical application, comprising of two electrodes particularly positioned within a liquid medium—to which high voltage can be applied in order to generate an electrical disruptive discharge, wherein at least one of the electrodes can travel between a first limit position that corresponds to the smallest possible distance between the two electrodes, and a second limit position, that corresponds to the largest possible distance between the electrodes, and that the apparatus is equipped with appliances for determining the first and/or the second limit position.

16. A procedure for the calibration of distances of a pair of submerged electrodes for the generation of shock waves by means of spark discharge under water, comprising the following steps of procedure; (i) movement of at least one of the electrodes up to a smallest possible distance between the electrodes; (ii) movement of at least one of the electrodes up to a largest possible distance between the electrodes; and (iii) simultaneous measurement of the travel distances.

17. The procedure according to claim No. 16, wherein a subsequent step of procedure is used to determine the remaining length of the electrodes and especially for determining the remaining number of shots in reference to the travel.

18. Procedure according to claim 17, wherein in a subsequent step a message with respect to the functionality of the pair of electrodes is read out.

19. Procedure for the generation of shock waves by means of under water spark discharge between two electrodes, comprising the steps of procedure.
(i) Execution of a calibration of distances and determination of the remaining number of shots of the electrodes;
(ii) Setting of the required energy level and the required number of shots;
(iii) Determination of the corresponding distance of the electrodes;
(iv) Inspection of the compatibility of the distance of the electrodes and the required number of shots with the remaining number of shots determined during the distance calibration, if required, an error message is to be generated or the procedure must be terminated;
(v) Setting of the distance between the two electrodes;
(vi) Discharges;
(vii) Repetition of the steps of procedure, starting with step (i) or step (ii).

20. Procedure according to claim No. 19, wherein after a discharge the readjustment of the distance between the two electrodes is executed.

21. Procedure according to claim No. 20, wherein the readjustment is carried out by means of the evaluation of:
the discharge curves, especially of the ignition delay periods, the extreme values of current and/or voltage, the attenuation response of the current and/or the voltage, the oscillating response of the discharge current and/or discharge voltage, the number of zero transitions of the discharge current and/or discharge voltage, the output and/or
the yield of pressure, especially the maximum pressure amplitude

22. Procedure according to claim 21, wherein a calibration of distances is carried out after a specifically defined number of discharges.
